(19) United States
(12) Patent Application Publication
(10) Pub. No.: US 2011/0166530 A1
(43) Pub. Date: Jul. 7, 2011

(54) HYALURONIC ACID DISPERSION, PRODUCTION AND USE

(75) Inventors: Christine Kreiner, Munchen (DE); Lidia Nachbaur, Berlin (DE)

(73) Assignee: S & V Technologies AG, Hennigsdorf (DE)

(21) Appl. No.: 12/309,467

(22) PCT Filed: Jul. 25, 2007
(86) PCT No.: PCT/EP2007/006609

§ 371 Date: Feb. 17, 2009

(51) Int. Cl. A61M 5/178; A61K 47/36; A61K 8/02

(52) U.S. Cl. 604/187; 514/777; 424/401

(57) ABSTRACT

The invention relates to a hyaluronic acid dispersion for use in aesthetic medicine and orthopedics, where the dispersed phase consists of particles of crosslinked hyaluronic acid, and the continuous phase essentially of linear hyaluronic acid.

### BACKGROUND OF THE INVENTION

Hyaluronic acid and preparations thereof have been known for a long time. Hyaluronic acid is an important constituent of human tissue and is present in the human body in the eye (vitreous humor), in bone joints and the epidermis. There are ca. 15 g of hyaluronic acid in the body of a person weighing 70 kg.

Hyaluronic acid is a macromolecular chain of disaccharides which consists of two glucose derivatives: D-glucuronic acid and N-acetyl-D-glucosamine. In the disaccharide, the glucuronic acid is linked to the N-acetylglucosamine, which in turn is β(1→4) joined to the next glucuronic acid in the polymeric chain. A chain here typically consists of 250-50 000 disaccharide units.

Hyaluronic acid and its preparations are used in the cosmetics sector, the pharmaceutical sector, and also the medical sector. In medicine, hyaluronic acid preparations are used for the treatment of joint complaints, in particular arthritic complaints. Here, the hyaluronic acid serves, injected directly into the joint, as joint lubricant, for improving the mobility of the joint apparatus. A further possible use of hyaluronic acid preparations in medicine is the field of ophthalmology, in particular cataract surgery.

In pharmacy, hyaluronic acid preparations serve as medicament carrier system since, on account of its high molecular weight and its particular chemical structure, hyaluronic acid is able to stabilize and quasiencapsulate chemical active substances in order, in so doing, to transport them, with the help of a suitable acceptable carrier material, into the inside of the cell, where the chemical substances develop their effect.

In the large field of aesthetics and cosmetics, hyaluronic acid is used in particular as a water store. Both in preparations to be used topically, and also in the case of the injection. By injecting hyaluronic acid directly under the skin, water depots are thus formed which expand like a sponge, and visibly “decrease” wrinkles on the outside, and when injected into lips, make these look full and well-shaped. Applied topically, hyaluronic acid increases the water retention capacity of the skin, as a result of which the transepidermal water loss (TEWL) is considerably reduced. The skin retains a fresh, youthful and tightened appearance and at the same time remains elastic and supple. Moreover, upon multiple application also in the case of hyaluronic acid preparations applied topically, small lines are reduced.

In aesthetics and cosmetics, hyaluronic acid, if applied topically, is in most cases used in solution or else in emulsified form. In order to achieve an adequate cosmetic effect, gel-based and emulsion-based preparations, however, are often not suitable since the other ingredients, such as preservatives, emulsifiers, solubilizers and surfactants, but also pigments and dyes, greatly catalyze the degradation reactions of hyaluronic acid, i.e. the cleavage of the molecular chains. Hyaluronic acid preparations therefore often only have an inadequate shelf-life, with mostly low effect.

An improvement with regard to the reduced chemical degradation of the molecules is offered by hyaluronic acid preparations which have hyaluronic acid chains with crosslinked structures. For this, the reactive side groups are reacted with suitable reactants, i.e. bridge molecules, as a result of which a three-dimensional network structure is formed. This in turn when tangled produces an extensive network structure with excellent water storage capacity.

For injecting hyaluronic acid, in most cases an aqueous hyaluronic acid solution is used. Hyaluronic acid is, as already explained, a macromolecule which, on account of its high molecular weight and its three-dimensional, tangled structure, has a high space requirement. The use concentrations are therefore quite low. The tangled, belt-like intertwined structure comes about as a result of the individual molecular sections entering into constructive interactions with one another, and, in the course of this, solvent molecules, generally water, being added on. On account of its chemical and physical structure, hyaluronic acid is a viscoelastic gel solution in water. The viscosity here is also considerably increased through crosslinking. In order to counteract premature degradation of the hyaluronic acid chains and to thereby extend the effectiveness of the hyaluronic acid, a sufficiently high degree of crosslinking is required. Crosslinked hyaluronic acid molecules are essentially more stable toward hydroxylation and/or chemical and enzymatic degradation by reactive molecules than noncrosslinked hyaluronic acid chains. Consequently, especially in medicine, aesthetics and cosmetics, where a lasting effect is decisive for the success of treatment, stabilized, crosslinked hyaluronic acid is exclusively used.

On account of the high viscosity of crosslinked hyaluronic acid, the applicability per injection for injecting under wrinkles, however, is only possible to a limited degree. For injection, the crosslinked hyaluronic acid is diluted in the dispersion to be applied in order to be able to use relatively thin needle internal diameters.

It is an object of the present invention to provide a hyaluronic acid preparation which is easy to inject and to distribute and is resistant to hydroxylation and chemical and enzymatic degradation and, moreover, in relation to standard commercial hyaluronic acid preparations, has a comparatively good, if not improved, mode of action, especially in relation to viscoaugmentation.

### DETAILED DESCRIPTION OF THE INVENTION

The object is achieved by a composition as disclosed in claim 1. Surprisingly, it has been found that a hyaluronic acid dispersion with a dispersed phase consisting of particles of crosslinked hyaluronic acid in a continuous phase of noncrosslinked hyaluronic acid achieves the stated object.

As already explained, hyaluronic acid is a macro-molecule. In particular, on account of its high molecular weight and its tangled structure, crosslinked hyaluronic acid is no longer able to dissolve in a surrounding solvent. This is therefore deemed to be a hyaluronic acid dispersion, i.e. a distribution of hyaluronic acid in the surrounding solvent. The majority of “solvent” or to put it better, dispersants, the continuous phase, consists of noncrosslinked hyaluronic acid. Unlike in the solvent water, in such a dispersion as is defined in claim 1 and the further embodiments, the behavior of the hyaluronic acid dispersion is viscoelastic, in particular thixotropic. This means that the dispersion liquefies upon the action of shear force, and solidifies again immediately after the action of the shear force has stopped and essentially returns to its original viscous structure.

Without being bound to the theory, it is assumed that the reason for this behavior has its origin as follows: the solvent water is a strongly polar molecule made of oxygen and hydrogen atoms. Hyaluronic acid also has polar groups in its chemical structure. However, in the case of the crosslinked structure, the polar anchor groups are reacted with a further molecule, meaning that the polar character is reduced. Consequently, in aqueous solution, the solubility of the crosslinked hyaluronic acid is considerably reduced. Like a drop of oil in an aqueous environment, the crosslinked hyaluronic acid in water also therefore becomes tangled together to an increased degree and forms insoluble sections in which the viscosity is extremely high. On account of the low solubility and therefore greatly pronounced tangling, such a preparation has a high viscosity and thus poorer distributability and injectability.

In contrast to this, in the invention, the effect of shear force leads to the viscosity of the dispersion according to the invention considerably decreasing at the moment of force impingement, and increasing again upon reduction of the shear force. This thixotropy of the hyaluronic acid dispersion according to the invention is decisive for the facilitated injectability, through which there is the option of using even smaller needle diameters upon injection. Furthermore, the hyaluronic acid dispersion according to the invention is best suited for soft tissue augmentation. Once injected under the skin, the viscosity increases again, so that in particular the wrinkle-filling effect occurs immediately after injection and thus the hyaluronic acid dispersion according to the invention is suitable for viscoaugmentation with long effectiveness and simultaneous excellent biocompatibility.

The fraction of crosslinked hyaluronic acid is about between 0.1 and 99.0% by weight, based on the total weight of the hyaluronic acid dispersion. Preferably, 50 to 95% by weight are used. Higher concentrations than 99% by weight lead to a viscosity increase, i.e. to a viscous liquid which does not differ significantly from pure crosslinked hyaluronic acid. Upon appropriate selection of the particle size, such a dispersion is likewise injectable. In order to supply adequately crosslinked hyaluronic acid to the site of action, about 50 to 95% by weight of crosslinked hyaluronic acid are best suited for also achieving a long-term effect. At lower fractions, the effectiveness time may be shorter.

The hyaluronic acid particles or dispersion droplets of the crosslinked hyaluronic acid preferably have a diameter of from about 80 to 300 μm. Since in particular transparent, gel-like dispersions are used for injection purposes, the particle diameter is preferably 80 to 300 μm. The lower limit of the particle diameter should be chosen with regard to the desired lastingly high effectiveness and the desired thixotropic properties.

Preferably, a crosslinked hyaluronic acid with a molecular weight of from about 0.8 to 3 million daltons is used. This is because low molecular weight hyaluronic acid molecules and thus also less crosslinked hyaluronic acid molecules are liable to accelerated degradation and are thus unable to develop a long-term effect.

The attached figure shows in diagrammatic representation the structure of the hyaluronic acid dispersion according to the invention. Crosslinked hyaluronic acid particles 1 are distributed in tangled linear hyaluronic acid 2. The tangled linear hyaluronic acid 2 also forms a protection (shell-in-shell) against degradation, as a result of which the desired lasting effectiveness following injection is achieved.

### Rheological Measurements on Working Examples

The rheological measurements were carried out using a shear stress controlled rheometer AR-G2 (Ta Instruments) at 25° C. The material functions were investigated using a cone plate geometry 0:40 mm, cone angle: 1°, gap: 28 μm) in a frequency range from ω=100 to 0.01 rad·s⁻¹ at deformations of γ=0.08% (example 1) and 0.1% (examples 2 and 3).

The oscillation experiment was only started following normal force constancy of the filling, i.e. after relaxation of the sample in the measurement gap. Table 1 gives an overview of the measurement conditions:

TABLE 1
Overview of the measurement conditions

| | |
|---|---|
| Instrument | TA AR-G2 |
| Cone-plate geometry | 40 mm diameter, gap distance 28 μm |
| Cone angle | 1° |
| Frequency range | 10⁻²-100 rad · s⁻¹ |
| Deformation | 0.08% (NaturalFace structure), |
| | 0.1% (NaturalFace contour) |
| Temperature | 25° C. |
| Data points | 10 per decade |

### Results:

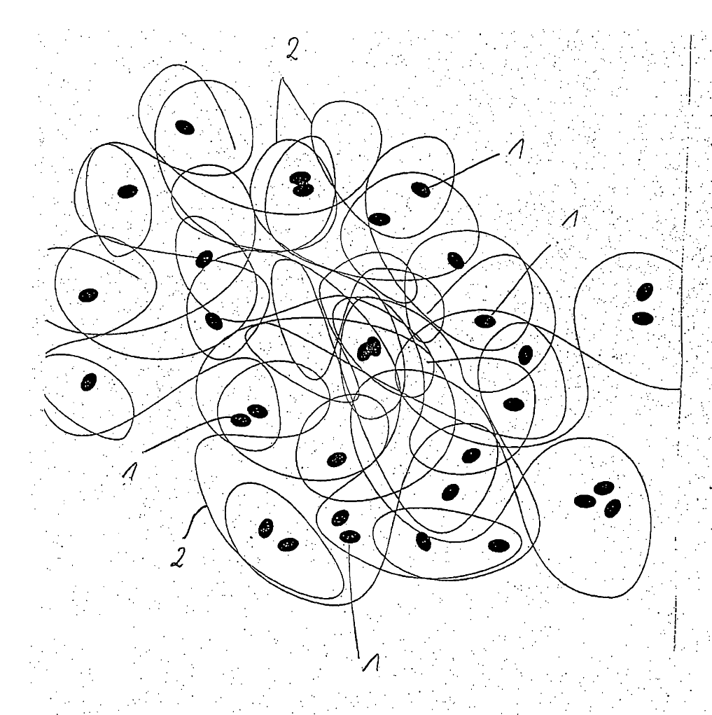

All three oscillator experiments were carried out in a frequency range 100 to 0.01 rad·s⁻¹. To determine the viscoelastic range of the particular sample, a so-called amplitude sweep was carried out in the run-up to each measurement. All of the samples have a plateau behavior over the entire measurement range with a slight decrease in the moduli toward the lower frequencies (halving of the storage modules over a measurement range of four decades) for the storage and/or loss modulus (FIGS. 1 to 3). According to the definition, this behavior is a feature of crosslinked samples. The complex viscosity I_{η}*I has a uniform gradient over the entire measurement range toward high frequencies, which likewise points to crosslinked samples. A zero-shear viscosity for none of the three samples can be ascertained for this reason. According to the definition for crosslinked samples, it can also never be achieved towards even smaller frequencies.

FIG. 3: Dependence of the complex oscillating viscosity η*, of the storage modulus G′ and of the loss modulus G″ on the applied radian frequency ω for example 3 (22 mg/ml crosslinked and 2 mg/ml uncrosslinked sodium hyaluronate solution).

The oscillator experiments of the samples of the three embodiments each have a plateau behavior of the moduli over the entire measurement range. Within this range, the behavior of the examples is viscoelastic on account of a quasi-permanent network. Storage and loss moduli of example 1 are about one decade below the two examples 2 and 3. The storage modulus G′ drops in example 1 from 276 Pa at 100 rad·s⁻¹ to a value of 111 Pa at 0.01 rad·s⁻¹. The development of the complex viscosity I_{η}*I in the case of example 1 has a similar course to both examples 2 and 3. The values of example 1 are here too about one decade lower compared to the two other examples. The reason for this is the low concentration of hyaluronic acid particles, the height of the plateau value being determined primarily by the fraction of crosslinked hyaluronic acid.

Examples 2 and 3 have a course which is identical within the scope of measurement accuracy both for the course of the two moduli G′ and G″, and also for the complex viscosity I_{η}*I. In the case of example 2, the storage modulus drops from 2439 Pa at a frequency of 100 rad·s⁻¹ to a value of 1327 Pa at 0.01 rad·s⁻¹ and in the case of example 3 from 2800 Pa at 100 rad·s⁻¹ to 1382 Pa at 0.01 rad·s⁻¹. The different particle size of the two examples (150 μm in the case of example 2 and 350 μm in the case of example 3) thus has only a very slight influence on the position of the two moduli.

The rheological measurement results show that the viscosity of the dispersion rapidly decreases even upon the slightest shear stress. Such a hyaluronic acid dispersion is therefore suitable in particular for injection, in particular for aesthetic medicine. On account of the low viscosity upon the action of shear force, the dispersion is liquefied such that canulas with a diameter of less than 0.2 mm internal diameter (e.g. 0.133 mm or 0.184 mm) are sufficient for injecting an effective amount of hyaluronic acid at the site of effect.

In addition, a hyaluronic acid dispersion as described above can be injected into an arthritic joint as arthrosis medicament. The fraction of crosslinked hyaluronic acid from the dispersion is able to store large amounts of water. As a result of this function, the hyaluronic acid dispersion can serve as joint lubricant replacement, as a result of which further wear of cartilage material is prevented or reduced, and the pain which is caused by the rubbing together of cartilage material that is insufficiently lubricated is lessened.

For such applications, a kit is provided which contains both a hyaluronic acid dispersion as claimed in claim 1, and also a suitable application device. This may, for example, be a syringe, the diameter of which is optimized in relation to the viscosity of the hyaluronic acid dispersion. In this connection, it is possible to use needle internal diameters of less than 0.2 mm, in particular from 18 to 22 gage.

Furthermore, a hyaluronic acid dispersion as described above can, however, also be used for aesthetic and cosmetic purposes. It is exceptionally suitable for being injected under wrinkles, in particular including the sensitive lips. By virtue of the reduced needle cross section of the injection needle, the puncture into the skin surface leaves behind virtually no detectable traces.

Moreover, hyaluronic acid dispersions, as are disclosed in claim 1 of the present invention, can also be used for topical application. For this, the hyaluronic acid dispersion is combined with customary cosmetic raw materials. Two examples of cosmetic preparations which comprise such a hyaluronic acid dispersion are described below. However, the examples here should not have a limiting effect, but merely be taken as examples.

### Example 1

### Hyaluronic Acid Cream

| | Amount |
|---|---|
| Raw material | [% by wt.] |
| Hyaluronic acid dispersion consisting of | 40.0 |
| 65% by wt. of crosslinked hyaluronic acid | |
| Water | 49.2 |
| Ethanol_{denat.} | 4.5 |
| PEG-16 Macadamia Glycerides | 5.0 |
| Sodium Polyacrylate | 0.1 |
| Sodium Cocoyl Apple Amino Acids | 0.5 |
| Phenoxyethanol | 0.3 |
| Sodium Methylparaben | 0.2 |
| Sodium Propylparaben | 0.2 |

### Preparation:

Water, PEG-16 macadamia glycerides and ethanol are initially introduced in a suitable container. With stirring at room temperature using a paddle stirrer, the preservatives (phenoxyethanol, sodium methylparaben and sodium propylparaben) are added. With vigorous stirring, the hyaluronic acid dispersion is added and stirring is continued until a homogeneous mixture is formed. Finally, the thickener (sodium polyacrylate) is added, whereupon the mixture thickens and forms a gel. The finished gel can be transferred to a suitable container, for example a dispenser or a can.

The resulting hyaluronic acid gel can be used as moisturizing gel for the treatment of dry areas of skin or else as wrinkle-smoothing gel. In particular, it develops its effect when it is applied underneath the lipcare stick after cleansing the face.

### Example 2

### Hyaluronic Acid Cream

| | Amount |
|---|---|
| Raw material | [% by wt.] |
| Hyaluronic acid dispersion consisting of | 35.0 |
| 70% by wt. of crosslinked hyaluronic acid | |
| Water | 45.2 |
| Polyglyceryl-3 Methylglucose Distearate | 3.0 |
| Beeswax | 2.7 |
| Silica | 0.6 |
| Inulin | 0.3 |
| Buxus Chinensis Oil | 12.4 |
| Imidazolidinyl Urea | 0.2 |
| Methylparaben | 0.3 |
| Propylparaben | 0.3 |

### Preparation:

In a suitable container, the water is initially introduced and stirred using a paddle stirrer at room temperature. The imidazolidinyl urea and the inulin are then added. As soon as the solution is clear, the hyaluronic acid dispersion is added and the mixture is stirred vigorously until homogeneous. The mixture is then heated to 45° C. In a separate container, the beeswax is weighed with the polyglyceryl-3 methyl-glucose distearate and the buxus chinensis oil and heated to 70° C. with stirring using a paddle stirrer. As soon as the waxes have melted and a homogeneous mixture has formed, the parabens are added. When both phases have reached their target temperature, the wax phase is slowly added to the water phase with vigorous stirring. The mixture is then cooled to 40° C. Finally, with further stirring, the silica is added and the cream is stirred until it has reached room temperature.

A cream of light consistency is obtained which is very highly suitable as day cream and thereby reduces face wrinkles.

1. A hyaluronic acid dispersion, characterized in that the dispersed phase consists of particles of crosslinked hyaluronic acid and the continuous phase consists essentially of linear hyaluronic acid.

2. The hyaluronic acid dispersion as claimed in claim 1, characterized in that the fraction of dispersed phase is about 0.1 to 90% by weight, based on the total hyaluronic acid dispersion.

3. The hyaluronic acid dispersion as claimed in claim 2, characterized in that the fraction of dispersed phase is about 50 to 75% by weight, based on the total hyaluronic acid dispersion.

4. The hyaluronic acid dispersion as claimed in claim 1, characterized in that the particle size of the crosslinked hyaluronic acid is about 80 to 300 pm.

5. The hyaluronic acid dispersion as claimed in claim 1, characterized in that the molecular weight of the crosslinked hyaluronic acid is about 0.8×106 to 3.0×106 daltons.

6. The hyaluronic acid dispersion as claimed in claim 1, characterized in that it is a clear gel.

7. The hyaluronic acid dispersion as claimed in claim 1, characterized in that it exhibits non-Newtonian behavior.

8. The hyaluronic dispersion as claimed in claim 7, characterized in that it exhibits thixotropic behavior.

9. The use of a hyaluronic dispersion as claimed in claim 1 for producing a preparation in cosmetics or aesthetics, in particular aesthetic medicine or in orthopedics, in particular as arthrosis medicament.

10. The use of a hyaluronic acid dispersion as claimed in claim 1, characterized in that it is applied topically or is injected.

11. The cosmetic composition comprising a hyaluronic acid dispersion, characterized in that the dispersed phase consists of particles of crosslinked hyaluronic acid and the continuous phase consists of linear hyaluronic acid.

12. The production of a cosmetic composition as claimed in claim 11, where the particles of crosslinked hyaluronic acid are dispersed in linear hyaluronic acid.

13. The use of a hyaluronic acid dispersion as claimed in claim 1 for producing a medicament.

14. A kit comprising a hyaluronic acid dispersion as claimed in claim 1 and an application device.

15. The kit as claimed in claim 14, characterized in that the application device is a syringe.

16. The kit as claimed in claim 12, characterized in that the syringe has an internal diameter of at least 0.2 mm and in particular of from 0.133 mm to 0.184 mm.

17. The use of a hyaluronic acid dispersion as claimed in claim 8, characterized in that it is applied topically or is injected.
